(19) United States
(12) Patent Application Publication
(10) Pub. No.: US 2025/0134712 A1
(43) Pub. Date: May 1, 2025

(54) THIN-FILM OCULAR IMPLANT AND DELIVERY TOOL IN OPEN OR MINIMALLY INVASIVE SURGICAL SITES

(71) Applicant: Avisi Technologies, Inc., Redwood City, CA (US)

(72) Inventor: Jeffrey Lorne Emery, Emerald Hills, CA (US)

(21) Appl. No.: 18/928,567

(22) Filed: Oct. 28, 2024

Related U.S. Application Data
(60) Provisional application No. 63/594,284, filed on Oct. 30, 2023.

(51) Int. Cl. A61F 9/007; A61B 17/34

(52) U.S. Cl. CPC A61F 9/00781; A61B 17/3468; A61F 2220/0016; A61F 2250/0097

(57) ABSTRACT

Described herein are insertion devices configured to achieve an efficient delivery and placement of a thin-film implant into a surgical site that is open or minimally invasive. An example insertion device includes a distal portion configured to enclose and protect a thin-film implant device during delivery and placement into a surgical site, wherein the distal portion includes a top half and a bottom half, and the thin-film implant device is placed between the top half and the bottom half; a support structure positioned between the top half and the bottom half and adjacent a proximal end of the thin-film implant device, wherein the top and bottom halves are retractable independently of one another.

### CROSS REFERENCE TO RELATED APPLICATION

This application claims priority to U.S. Provisional Patent Application No. 63/594,284 filed on Oct. 30, 2023, entitled “THIN-FILM OCULAR IMPLANT AND DELIVERY TOOL IN OPEN OR MINIMALLY INVASIVE SURGICAL SITES,” the contents of which are incorporated by reference herein in their entirety.

### BACKGROUND

Thin-film implants may present unique challenges for delivery or placement with conventional surgical tools in that these surgical tools may damage the thin-film implant, be unable to grip the implant in the proper orientation, or not maintain the implant in a planar manner. Conventional surgical tools may not be able to support the implant without damage as it is delivered, particularly if the delivery is minimally invasive and requires pushing through small incisions or tunnels in the tissues. Conventional surgical tools may also damage surrounding tissue during implantation, diminishing the minimally-invasive benefits of thin film implants. Further, conventional tools may not achieve final proper placement of the implant, such as being flat, without wrinkles, creases or folding at the surgical site, without significant subsequent manipulation by a variety of other tools.

Accordingly, there is a need to provide a device, system and method configured to achieve an efficient delivery and placement of at least one thin-film implant into a surgical site that is open or minimally invasive (e.g., an ocular surgical site).

### SUMMARY

Described herein are insertion devices, systems and methods configured to manipulate and deliver at least one thin-film implant into a surgical site that is open or minimally invasive (e.g., an ocular surgical site). In one preferred embodiment, a treatment tool may be configured to facilitate placement of an aqueous shunt device within the eye to lower intraocular pressure for the treatment of glaucoma.

Among other features, the present disclosure provides a convenient, reliable, and secure system having a tool that is uniquely matched to a thin-film or small implant to ensure its successful installation into an open or minimally invasive surgical site. The systems and methods of the present disclosure reduce the variability and risks associated with user applied forces through traditional tools such as forceps as well as enabling low profile or even minimally invasive delivery options for an implant.

In one aspect, the present disclosure relates to an insertion device or tool, comprising: a distal portion configured to enclose and protect a thin-film implant device during delivery and placement into a surgical site, wherein the distal delivery tool portion includes a top half and a bottom half, and the thin-film implant device is placed between the top half and the bottom half; an internal support structure positioned between the top half and the bottom half and adjacent to a proximal end of the thin-film implant device, wherein the top and bottom halves are retractable independently of one another and the internal support structure via a user-operated handle.

In one embodiment, the insertion device may further comprise a handle portion, and an external marker placed at a location to communicate important information to the user, such as how deep to insert the tool into the surgical site.

According to some implementations, the handle portion of the insertion device may be configured to include a component to retract the top half of the distal portion followed by a retraction of the bottom half in a sequential order, wherein the component may include at least one of a lever, a button, a switch, and a slider. In an alternate embodiment, a wheel or scrolling mechanism may be implemented on the insertion device, such that the user can have full control of how quickly the top and bottom halves of the insertion device retract.

In yet another embodiment, the handle portion of the insertion device may be configured to include two levers to control retractions of the top half and the bottom half of the distal portion independently.

Further, the thin-film implant device may be configured to prevent axial and/or lateral movements of the thin-film implant device after implantation. In one embodiment, the thin-film implant device may have an outer surface that includes a plurality of lateral notches respectively along some or all of the perimeters of the thin-film implant device to promote fixation via tissue integration. In this way the thin-film implant device may not require sutures for preventing migration post-implantation.

Alternatively, the top and/or bottom outer surface(s) of the thin-film implant device may be configured to include micro-barbs or similar features configured to promote retention.

The insertion device may also include a curvature or shape to match the curved nature of the ideal implantation site. For example, the distal portion of the insertion device may be curved in order to avoid being obstructed by, making contact with, getting caught on, or harming other tissues as the user navigates the insertion device to the implantation site.

In addition, the insertion device may be flexible and able to be deflected to a custom shape by a user in order to accommodate the specific anatomy of the patient as they navigate to the implantation site. For example, the orbital bones of the eye or nasal ridge may require unique curvature from patient to patient in order for the user to reach the target implant location.

According to another embodiment, the distal portion of the insertion device may be made of transparent materials to allow a direct visualization of the thin-film implant device.

According to another embodiment, either or both the thin-film implant device and the insertion device may have a visual marker at one end to indicate the proper depth of insertion within the surgical site.

According to another embodiment, the tip of the distal end of the insertion device may be beveled to facilitate penetration of tissues into the target surgical site.

Additional features and advantages are described in, and will be apparent from, the following Detailed Description and the Figures. The features and advantages described herein are not all-inclusive and, in particular, many additional features and advantages will be apparent to one of ordinary skill in the art in view of the figures and description. Also, any particular embodiment does not have to have all of the advantages listed herein and it is expressly contemplated to claim individual advantageous embodiments separately. Moreover, it should be noted that the language used in the specification has been selected principally for readability and instructional purposes, and not to limit the scope of the inventive subject matter.

### BRIEF DESCRIPTIONS OF THE DRAWINGS

The present disclosure will become more fully understood from the detailed description and the accompanying drawings, wherein:

FIG. 1 is a perspective view of a device according to one embodiment.

FIG. 2 is a close-up view of the device according to section A identified in FIG. 1.

FIG. 3 is a cross-sectional view of the device shown along line III-III in FIG. 2.

FIG. 4 is a perspective view of a device according to another embodiment.

FIG. 5A is a portion of a cross-sectional view of section A of the device shown in FIG. 4 according to one embodiment.

FIG. 5B is a portion of a cross-sectional view of section A of the device shown in FIG. 4 according to one embodiment.

FIG. 5C is a portion of a cross-sectional view of section A of the device shown in FIG. 4 according to one embodiment.

FIG. 6 is a diagram of a treatment device implanted within a supraciliary or suprachoroidal space, according to an example embodiment of the present disclosure.

FIG. 7A is a diagram of a treatment device loaded in an insertion device being inserted in supraciliary space of an eye, according to an example embodiment of the present disclosure.

FIG. 7B is a diagram of a top portion of a treatment device loaded in an insertion device retracting into a user-operated handle, according to an example embodiment of the present disclosure.

FIG. 7C is a diagram of a bottom portion of a treatment device loaded in an insertion device retracting into a user-operated handle, according to an example embodiment of the present disclosure.

FIG. 7D is a diagram of an insertion device being removed from a surgical site, leaving a treatment device in place, according to an example embodiment of the present disclosure.

FIG. 8 is a diagram of a treatment device with lateral notches, according to an example embodiment of the present disclosure.

FIG. 9 is a diagram of an insertion device with the treatment device of FIG. 8 preloaded therein, according to an example embodiment of the present disclosure.

FIG. 10 is a diagram of the insertion device of FIG. 9 during a first step of deployment, according to an example embodiment of the present disclosure.

FIG. 11 is a diagram of the insertion device of FIG. 9 during a second step of deployment, according to an example embodiment of the present disclosure.

FIG. 12 is a diagram of a fully deployed treatment device, according to an example embodiment of the present disclosure.

FIG. 13 illustrates an insertion device having a straight shaft and a plurality of notches implemented on a lower portion of a flexible tip portion of the insertion device, according to an example embodiment of the present disclosure.

FIG. 14 illustrates an insertion device having a curved shaft and a plurality of notches implemented on a lower portion of a flexible tip portion of the insertion device when the insertion device is inserted into eye tissue, according to an example embodiment of the present disclosure.

FIG. 15 illustrates multiple notches implemented on a bottom portion of an insertion device before flexing, according to an example embodiment of the present disclosure.

FIG. 16 illustrates maximum flexing of multiple notches implemented on a bottom portion of an insertion device, according to an example embodiment of the present disclosure.

FIG. 17 illustrates an insertion device including a lumen and a guidewire for positioning a treatment device at a surgical site in an eye, according to an example embodiment of the present disclosure.

### DETAILED DESCRIPTION

Various aspects of the present disclosure will be described with reference to the drawings, wherein like reference numerals are used to refer to like elements throughout. In the following description, for purposes of explanation, numerous specific details are set forth in order to promote a thorough understanding of one or more aspects of the present disclosure. It may be evident in some or all instances, however, that any aspects described below can be practiced without adopting the specific design details described below.

It should be appreciated that the device, system and method of the present disclosure may be utilized in any one or more medical or surgical procedures that involve fragile thin-film like implants such as, for example cardiac surgery, neurosurgery, plastic surgery, anastomosis procedures, non-surgical procedures, endoscopic procedures, non-invasive procedures, invasive procedures, port-access procedures, fluoroscopic procedures, beating heart surgery, vascular surgery, neurosurgery, electrophysiology procedures, diagnostic and therapeutic procedures, ablation procedures, ablation of arrhythmias, endovascular procedures, treatment of one or more organs and/or vessels, cardiograms, pharmacological therapies, drug delivery procedures, delivery of biological agents, gene therapies, cellular therapies, cancer therapies, radiation therapies, genetic, cellular, tissue and/or organ manipulation or transplantation procedures, coronary angioplasty procedures, placement or delivery of coated or uncoated stents, placement of cardiac reinforcement devices, placement of cardiac assistance devices, atherectomy procedures, atherosclerotic plaque manipulation and/or removal procedures, emergency procedures, cosmetic procedures, reconstructive surgical procedures, biopsy procedures, autopsy procedures, surgical training procedures, birthing procedures, congenital repair procedures, and medical procedures that require manipulation and delivery of one or more fragile thin-film like implant into a surgical site.

In one embodiment, as will be described fully below, the present disclosure relates to holding, placement and delivery of a thin-film based ocular implant such as for treatment of glaucoma. A glaucoma drainage implant is a small device (i.e., a thin-film device) placed in an eye of a patient to treat glaucoma. Most glaucoma patients have abnormally high intraocular pressure (IOP) due to the patient's inability to drain excessive aqueous humor from the anterior chamber of the eye through the trabecular meshwork. If not reduced with adequate treatment, high IOP will continuously damage the optic nerve as the disease progresses, leading to loss of vision or even total blindness. During a glaucoma implant surgery, a tiny drainage hole may be made in the sclera of the patient's eye (the white part of the eye). This opening allows fluid to drain out of the eye under the delicate membrane covering the eyeball known as the conjunctiva. Locally applied medications or injections may be used to keep the hole open and a thin-film glaucoma drainage device is positioned on the outside of the eye under the conjunctiva to drain excessive fluid out of the eye and into a place where the capillaries and lymphatic system of the patient reabsorb it back into the body, thereby lowering the intraocular pressure.

To reduce inflammation, scarring, operative time, risks of adverse events, and post-operative patient discomfort, it is desired to make as small as incision of the target implantation site (such as in the cornea to access the supraciliary space) as possible, ideally less than 2 millimeters (“mm”) wide×1 mm tall. However, the treatment device, in many embodiments, has a width between 1 and 10 millimeters, preferably around 2 mm wide, and a height of up to 1 mm, preferably 0.1 mm, to provide adequate drainage of aqueous humor from an anterior chamber of a patient's eye. Ideally, this means that the delivery tool is minimally wider and taller than the implant itself.

To enable a minimally invasive insertion, the treatment device is preloaded into an insertion device. For delivery of an aqueous drainage implant to the supraciliary space, access is obtained through a small incision in the cornea opposite to the target site. After the insertion device passes across the anterior chamber, the insertion device tip penetrates the supraciliary space. The insertion device then dispenses the treatment device so that it rests flat or nearly flat within the supraciliary pocket.

As used throughout, ranges are used as shorthand for describing each and every value that is within the range. Any value within the range can be selected as the terminus of the range. In addition, all references cited herein are hereby incorporated by reference in their entireties. In the event of a conflict in a definition in the present disclosure and that of a cited reference, the present disclosure controls.

The description of illustrative embodiments according to principles of the present invention is intended to be read in connection with the accompanying drawings, which are to be considered part of the entire written description. In the description of embodiments of the invention disclosed herein, any reference to direction or orientation is merely intended for convenience of description and is not intended in any way to limit the scope of the present invention. Relative terms such as “lower,” “upper,” “horizontal,” “vertical,” “above,” “below,” “up,” “down,” “top,” and “bottom” as well as derivatives thereof (e.g., “horizontally,” “downwardly,” “upwardly,” etc.) should be construed to refer to the orientation as then described or as shown in the drawing under discussion. These relative terms are for convenience of description only and do not require that the structure be constructed or operated in a particular orientation unless explicitly indicated as such.

Terms such as “attached,” “affixed,” “connected,” “coupled,” “interconnected,” and similar refer to a relationship wherein structures are secured or attached to one another either directly or indirectly through intervening structures, as well as both movable or rigid attachments or relationships, unless expressly described otherwise. Moreover, the features and benefits of the invention are illustrated by reference to the exemplified embodiments. Accordingly, the invention expressly should not be limited to such exemplary embodiments illustrating some possible non-limiting combination of features that may exist alone or in other combinations of features; the scope of the invention being defined by the claims appended hereto.

Unless otherwise specified, all percentages and amounts expressed herein and elsewhere in the specification should be understood to refer to percentages by weight. The amounts given are based on the weight of the material. According to the present application, the term “about” means+/−5% of the reference value. According to the present application, the term “substantially free” means less than about 0.1 wt. % based on the total of the referenced value.

A “subject” herein may be a human or a non-human animal, for example, but not by limitation, rodents such as mice, rats, hamsters, and guinea pigs; rabbits; dogs; cats; sheep; pigs; goats; cattle; horses; and non-human primates such as apes and monkeys, etc.

### Treatment Device Embodiment

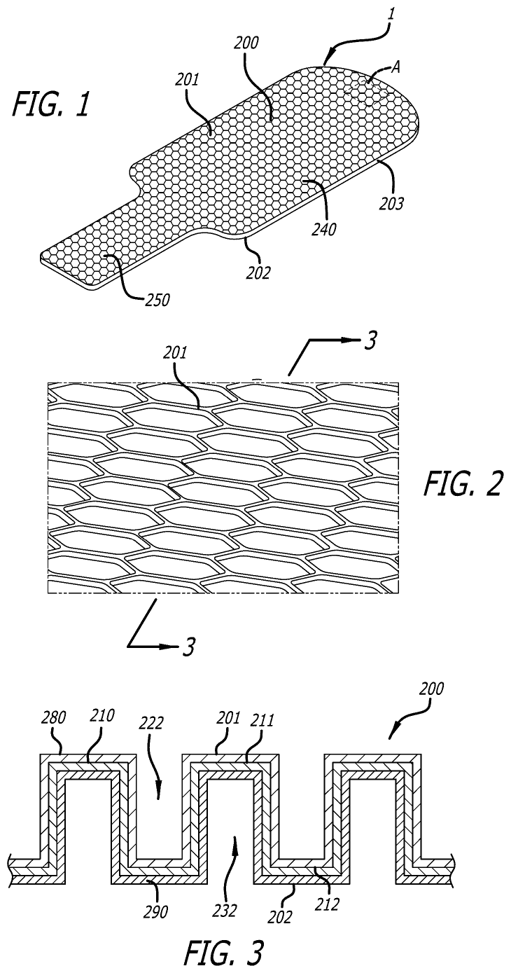

Referring to FIGS. 1-3, a treatment device 1 includes a plate structure 200, or simply plate, having a first major exposed surface 201 opposite a second major exposed surface 202 as well as side surface 203 extending there-between. The plate structure 200 can comprise an extension portion 250 and a main body portion 240.

The plate structure 200 can be formed of any material with appropriate characteristics for implantation and treatment. In some embodiments, the plate structure 200 can be formed of a metal, polymer, ceramic (e.g., aluminum oxide), other composite material, or a combination thereof. Metals can include, but are not limited to aluminum, titanium, zinc, platinum, tantalum, copper, nickel, rhodium, gold, silver, palladium, chromium, iron, indium, ruthenium, osmium, tin, iridium, or combinations, and alloys thereof. In some embodiments, alloys can include steel and nickel titanium such as Nitinol.

Polymers or polymer materials used to form plate structure 200 can include any of the polymers described herein.

Composites such as silicon composites can also be used. In one embodiment, a composite can include silicon nitride (Si₃N₄). The silicon nitride can have any known crystalline structure such as, but not limited to, trigonal α-Si₃N₄, hexagonal β-Si₃N₄, or cubic γ-Si₃N₄.

The plate structure 200, or plate, can have a thickness ranging from about 1 nm to about 1,000 nm, from about 1 nm to about 500 nm, from about 1 nm to about 400 nm, from about 100 nm to about 1,000 nm, from about 200 nm to about 1,000 nm, from about 300 nm to about 1,000 nm, from about 400 nm to about 1,000 nm, from about 1 nm to about 900 nm, from about 1 nm to about 800 nm, from about 1 nm to about 700 nm, from about 1 nm to about 600 nm, from about 300 nm to about 500 nm, from about 300 nm to about 600 nm, from about 400 nm to about 600 nm, from about 200 nm to about 600 nm, from about 200 nm to about 500 nm, or from about 50 nm to about 800 nm.

The plate structure 200 may comprise a multi-directional plate 210 comprising a first major surface 211 opposite a second major surface 212. The multi-directional plate 210 may form a plurality of topographical features (for example, a repeating honeycomb pattern) on each of the first major surface 211 and the second major surface 212. Each of the first and second topographies may independently comprise a plurality of channels 232 and/or a plurality of open-cells 222.

The plurality of channels 232 may be interconnected and can form a network of channels. The channels may be open or closed, allowing fluid to readily enter each channel of plurality of channels 232 and flow through it. The network may comprise intersecting channels in any suitable configuration to best help promote the flow of fluid across the plate structure 200 via the plurality of channels 232. In one embodiment, the channels 232 may be configured to form hexagonal patterns. Once treatment device 1, illustrated in FIG. 1, is implanted, fluid (e.g., aqueous humor) may be driven by a pressure gradient to flow through the channels and across the surface of plate structure 200.

In some embodiments, the channels 232 can include a ribbing pattern. The ribbing pattern and/or the geometry of the channels in the plate can be varied based on different severities of disease (e.g., mild, moderate, or severe glaucoma). In one embodiment, larger or smaller channels can be used to decrease intraocular pressure by different amounts. Changing intraocular pressure by a lower amount can decrease risk of hypotony (a condition that can exist if intraocular pressure is reduced too much) and increase efficacy at lowering pressure to a target level. In some embodiments, a device as described herein with smaller channels can decrease flow and decrease risk of hypotony. Likewise, larger channels can increase flow and allow the device to reduce intraocular pressure to a lower level.

The plate structure 200 may further comprise a first coating 280 applied to the first major surface 211 of the multi-directional plate 210. The first coating 280 may conform to the first topography of the first major surface 211 of the multi-directional plate 210. In other embodiments, the first coating 280 may form a topography that does not conform to the first topography of the first major surface 211 of the multi-directional plate 210.

The first coating 280 may have a thickness ranging from about 0.1 μm to about 10 μm or about 0.1 μm to about 2 μm—including all thickness and sub-ranges there-between. In one embodiment, the thickness is between about 0.4 μm (400 nm) and 0.6 μm (600 nm). In one embodiment, the thickness is about 0.4 μm (400 nm). In other embodiments, the thickness is between about 1 μm and about 5 μm, between about 1 μm and about 3 μm, between about 2 μm and about 5 μm, or between about 2 μm and about 4 μm. In one embodiment, the thickness is about 2 μm.

The plate structure 200 may further comprise a second coating 290 applied to the second major surface 212 of the multi-directional plate 210. The second coating 290 may conform to the plurality of surface features on the second major surface 212 of the multi-directional plate 210. In other embodiments, the second coating 290 may form a topography that does not conform to the second topography of the second major surface 212 of the multi-directional plate 210.

The second coating 290 may have a thickness ranging from about 0.1 μm to about 10 μm or about 0.1 μm to about 1 μm-including all thickness and sub-ranges there-between. In one embodiment, the thickness is between about 0.4 μm (400 nm) and 0.6 μm (600 nm). In one embodiment, the thickness is about 0.4 μm (400 nm). In other embodiments, the thickness is between about 1 μm and about 5 μm, between about 1 μm and about 3 μm, between about 2 μm and about 5 μm, or between about 2 μm and about 4 μm. In one embodiment, the thickness is about 2 μm.

In some embodiments, the plate structure 200 may comprise only the first coating 280—i.e., no second coating. In other embodiments, the plate structure 200 may comprise only the second coating 290—i.e., no first coating. In other embodiments, the plate structure 200 may comprise the first coating 280 and the second coating 290, whereby the first and second coatings overlap to fully encapsulate the multi-directional plate 210. In such embodiments, the side surface 203 of the plate structure 200 may comprise at least one of the first coating 280 and the second coating 290.

In some embodiments, the first and second coating, and any edge coating, can be thicker than the plate itself. In some embodiments, the coating thickness can be one, two or three orders of magnitude thicker than the plate structure. However, in other embodiments, the plate can be thicker than each coating or the additive thickness of the two coatings.

Coatings described herein can be applied by any suitable deposition method, such as but not limited to, physical vapor deposition, chemical vapor deposition, atomic layer deposition, spray coating, spin coating, self-assembly, dip coating, or brushing.

The first coating 280 may be applied to the first major surface 211 by any suitable deposition method. In a non-limiting example, the first coating 280 may be applied to the first major surface 211 by chemical vapor deposition, physical vapor deposition, or plasma-enhanced chemical vapor deposition. In another non-limiting example, the first coating 280 may be applied to the first major surface 211 by atomic layer deposition. In another non-limiting example, the first coating 280 may be applied to the first major surface 211 by spray coating. In another non-limiting example, the first coating 280 may be applied to the first major surface 211 by dip coating. In another non-limiting example, the first coating 280 may be applied to the first major surface 211 by brushing.

The second coating 290 may be applied to the second major surface 212 by any suitable deposition method. In a non-limiting example, the second coating 290 may be applied to the second major surface 212 by chemical vapor deposition, physical vapor deposition, or plasma-enhanced chemical vapor deposition. In another non-limiting example, the second coating 290 may be applied to the second major surface 212 by atomic layer deposition. In another non-limiting example, the second coating 290 may be applied to the second major surface 212 by spray coating. In another non-limiting example, the second coating 290 may be applied to the second major surface 212 by dip coating. In another non-limiting example, the second coating 290 may be applied to the second major surface 212 by brushing.

The first coating 280 may be the same as the second coating 290. The first coating 280 and the second coating 290 may be different. The first coating 280 may be hydrophilic. The first coating 280 may be hydrophobic. The first coating 280 may be lipophilic. The first coating 280 may be lipophobic. The second coating 290 may be hydrophilic. The second coating 290 may be hydrophobic. The second coating 290 may be lipophilic. The second coating 290 may be lipophobic. Each of the first and second coatings 280, 290 may independently be continuous. Each of the first and second coatings 280, 290 may independently be discontinuous. In some embodiments, the first and second coatings 280, 290 may both be hydrophobic. In some embodiments, the first and second coatings 280, 290 may both be hydrophilic. In some embodiments, the first and second coatings 280, 290 may both be lipophilic or lipophobic.

The first coating 280 may be organic. The first coating 280 may be inorganic. The second coating 290 may be organic. The second coating 290 may be inorganic.

In some embodiments, the first coating 280 is hydrophilic and the second coating 290 is hydrophobic. In some embodiments, the first coating 280 is hydrophilic and the second coating 290 is hydrophilic. Having at least one of the first and/or second coating 280, 290 be hydrophobic may help prevent the treatment device 1 from inadvertently sticking to tissue during implantation.

In some embodiments, a purpose of a first and/or second coating is to increase the toughness of the device. Also, a first and/or second coating can increase biocompatibility of the device and/or decrease scarring by decreasing tissue and/or fibroblast adhesion. In some embodiments, the coatings described herein are hydrophobic and decrease tissue adhesion. In some embodiments, tissue adhesion can be reduced by greater than about 10%, greater than about 60%, greater than about 70%, greater than about 80%, greater than about 90%, greater than about 95%, greater than about 96%, greater than about 97%, greater than about 98%, or greater than about 99% when compared to an uncoated plate.

In a non-limiting embodiment, the first and/or second coating may comprise a polymer, such as a parylene polymer (poly(para-xylylene)) or a derivative thereof. In other embodiments, the first and/or second coating can include aluminum oxide, a biocompatible film, a porous coating, or a lubricious coating. In one embodiment, the parylene polymer is a chlorine modified poly(para-xylylene), or a fluorine modified poly(para-xylylene). In one embodiment, the parylene polymer can be parylene C, parylene D, parylene N, a derivative thereof or a combination thereof. In other embodiments, the first and/or second coating can include aluminum oxide.

In other embodiments, other polymer(s) can be used in addition to, in combination with, or instead of a parylene polymer and/or aluminum oxide. In some embodiments, other polymeric materials can include, but are not limited to rubber, synthetic rubber, silicone polymers, thermoplastics, thermosets, polyolefins, polyisobutylene, acrylic polymers, ethylene-co-vinylacetate, polybutylmethacrylate, vinyl halide polymers (for example, polyvinyl chloride), polyvinyl ethers (for example, polyvinyl methyl ether), polyvinylidene halides, polyacrylonitrile, polyvinyl ketones, polyvinyl aromatics, polyvinyl esters, acrylonitrile-styrene copolymers, ABS resins, ethylene-vinyl acetate copolymers, polyamides (for example, Nylon 66 and polycaprolactam), alkyd resins, polycarbonates, polyoxymethylenes, polyimides, polyethers, epoxy resins, polyurethanes, rayon, cellulose, cellulose acetate, cellulose butyrate, cellulose acetate butyrate, cellophane, cellulose nitrate, cellulose propionate, cellulose ethers, carboxymethyl cellulose, polytetrafluororethylene (for example, Teflon), poly(ether-ether-ketone), poly lactides such as PLA, PLGA, PLLA, derivatives thereof, or combinations thereof.

The resulting treatment device 1 may comprise the first plurality of open-cells 222 present on the first major exposed surface 201 of the plate structure 200, wherein the first plurality of open-cells 222 are hydrophilic due to the presence of the first coating 280. The resulting treatment device 1 may comprise the second plurality of channels 232 present on the second major exposed surface 202 of the plate structure 200, wherein the second plurality of channels 232 are hydrophilic due to the presence of the second coating 290. As discussed, the hydrophilic channels may promote fluid flow through the channels after the treatment device 1 has been implanted into a subject's eye.

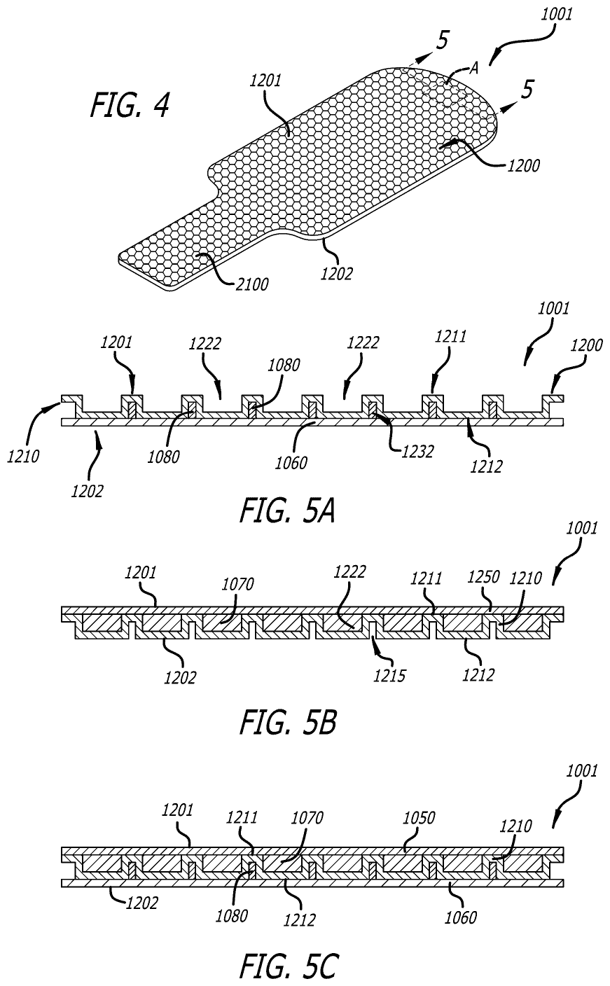

Referring to FIGS. 4, 5A, 5B, and 5C, generally, a treatment device 1001 is illustrated in accordance with another embodiment. The treatment device 1001 is similar to the treatment device 1 except as described herein below. The description of the treatment device 1 above generally applies to the treatment device 1001 described below except with regard to the differences specifically noted below. A similar numbering scheme will be used for the treatment device 1001 as with the treatment device 1 except that a “1000” series of numbering will be used.

The treatment device 1001 comprises a plate structure 1200 having a first major exposed surface 1201 that is opposite a second major exposed surface 1202. The plate structure 1200 may comprise a multi-directional plate 1210 comprising a first major surface 1211 opposite a second major surface 1212. The multi-directional plate 1210 may form a plurality of topographical features (for example, a repeating honeycomb pattern) on each of the first major surface 1211 and the second major surface 1212. Each of the first and second topographies may independently comprise a plurality of channels 1232 and/or a plurality of open-cells 1222.

Referring now to FIG. 5B, the plate structure 1200 may comprise a first drug-treatment delivery component 1070 present in the open voids created by the first topography formed by the first exposed surface 1211 of the multi-directional plate 1210. Specifically, the first delivery component 1070 may be present in the open voids created by the plurality of open-cells 1222 of first topography formed by the first major surface 1211 of the multi-directional plate 1210.

The first drug-treatment delivery component 1070 may comprise one or more active agents such as, but not limited to therapeutic and/or pharmacological components. The first drug-treatment delivery component 1070 may occupy some, all, or substantially all of the free volume present in the plurality of open-cells 1222 formed by the first topography.

In other embodiments, active agents can include any compound or drug having a therapeutic effect in a subject. Non limiting active agents include anti-proliferatives including, but not limited to, macrolide antibiotics including FKBP-12 binding compounds, estrogens, chaperone inhibitors, protease inhibitors, protein-tyrosine kinase inhibitors, leptomycin B, peroxisome proliferator-activated receptor gamma ligands (PPARD), hypothemycin, nitric oxide, bisphosphonates, epidermal growth factor inhibitors, antibodies, steroids, proteasome inhibitors, antibiotics, anti-inflammatoirenti-sense nucleotides, transforming nucleic acids, messenger ribonucleic acids, IOP lowering drugs, prostaglandins, cytostatic compounds, toxic compounds, anti-inflammatory compounds, chemotherapeutic agents, analgesics, antibiotics, protease inhibitors, statins, nucleic acids, polypeptides, growth factors and delivery vectors including recombinant micro-organisms, cells, stem cells, liposomes, anti-metabolites such as mitomycin-C, combinations thereof, prodrugs thereof, pharmaceutical salts thereof, derivatives thereof, and the like.

The treatment device 1001 may further comprise a first coating 1050 applied to a first major surface 1211 of the multi-directional plate 1210. The first coating 1050 may cover both a first major surface 1211 of the multi-directional plate 1210 as well as a first drug-treatment delivery component 1070 that is present in the plurality of open-cells 1222 formed into the first major surface 1211 of the multi-directional plate 1210. The first coating 1050 may be in the form of a continuous film. The first coating 1050 may be flat. In other embodiments, the first coating 1050 may be conformal to the underlying pattern formed by the multi-directional plate 1210 and the first delivery component 1070.

Referring now to FIG. 5A, the plate structure 1200 may comprise a second drug-treatment delivery component 1080 present in the open voids created by the second topography formed by the second exposed surface 1212 of the multi-directional plate 1210. Specifically, the second delivery component 1080 may be present in the open voids created by the open-channels 1232 of the second topography formed by the second major surface 1212 of the multi-directional plate 1210. The second delivery component 1080 may be the same or different from the first delivery component 1070.

The second drug-treatment delivery component 1080 may comprise one or more therapeutic and/or pharmacological components-including but not limited to anti-inflammatory agents, steroids, antibiotics, analgesics. The second delivery component 1080 may occupy some, all, or substantially all of the free volume present in the channels 1232 formed by the first topography.

The treatment device 1001 may further comprise a second coating 1060 applied to a second major surface 1212 of the multi-directional plate 1210. The second coating 1060 may cover both the second major surface 1212 of the multi-directional plate 1210 as well as the second delivery component 1080 that is present in the open-channels 1232 formed into the second major surface 1212 of the multi-directional plate 1210. The second coating 1060 may be in the form of a continuous film. The second coating 1060 may be flat. In other embodiments, the second coating 1060 may be conformal to the underlying pattern formed by the multi-directional plate 1210 and the second delivery component 1080.

The second coating 1060 may be the same or different than the first coating 1050. For each of the first and the second coatings 1050, 1060, the resulting film may be formed from a slow-release material that dissolves slowly after exposure to aqueous humor or other biological fluids, thereby releasing the first delivery component 1070 from the channels 1232 of the treatment device 1001 after it has been implanted into a subject.

Referring now to FIG. 5C, in other embodiments, the treatment device 1001 may comprise both the first and the second drug-treatment delivery components 1070, 1080, as well as the first and the second coatings 1050, 1060 to encapsulate the first and second delivery components 1070, 1080.

In other embodiments, the plate structure 1200 may comprise at least one of the first coating 1050 and/or the second coating 1060 without the presence of the first and/or second delivery components 1070, 1080. In such embodiments, the first coating 1050 and/or the second coating 1060 may form a film that covers the plurality of open-cells 1222 and/or the open channels 1232 created by the multi-directional plate.

The presence of the films resulting from the first and/or the second coating 1050, 1060 may enhance the overall strength of the resulting treatment device. Specifically, layered structure(s) of the films formed by the first and second coatings 1050, 1060, which are bonded to the first and second major surfaces 1211, 1212 of the multi-directional plate 1210, provide added mechanical integrity to the resulting treatment device.

Beyond achieving the baseline flexibility to conform to curvature of the eye, the addition of the first and/or second coatings 1050, 1060 may provide a mechanism that allows the overall treatment device to match the elastic modulus of surrounding tissues (e.g., conjunctival and scleral tissues) to maximize biocompatibility or biointegration. Findings in brain implant research confirm that the flexibility of implants in soft tissue improves compliance of the implant with microscale movements of surrounding tissue and reduces tissue displacement and trauma as well as facilitates implantation of the treatment device.

### Treatment Device Insertion Embodiment

In accordance with aspects of the present disclosure, certain selected locations within the eye may be used to shunt aqueous fluid and help prevent buildup of excessive ocular pressure. For example, the supraciliary space is a physiological route for aqueous humor outflow located anteriorly between the outer surface of the ciliary body and the internal surface of the sclera. Posteriorly, the suprachoroidal space is located between the choroid and the internal surface of the sclera. These spaces have been targeted as potential pathways for glaucoma treatment. The traditional subconjunctival space, in certain situations, may be affected by several limitations such as poor aqueous drainage (which may require additional treatment with medication or surgical intervention), poor cosmesis, a lifetime risk for endophthalmitis, and an unpredictable wound healing response. Because of these limitations, the supraciliary space and suprachoroidal space have gained growing interest as potential locations for receiving aqueous fluid drainage devices, as both spaces may offer a non-bleb option for aqueous absorption that is advantageous to certain customer types who would prefer a more minimally invasive approach.

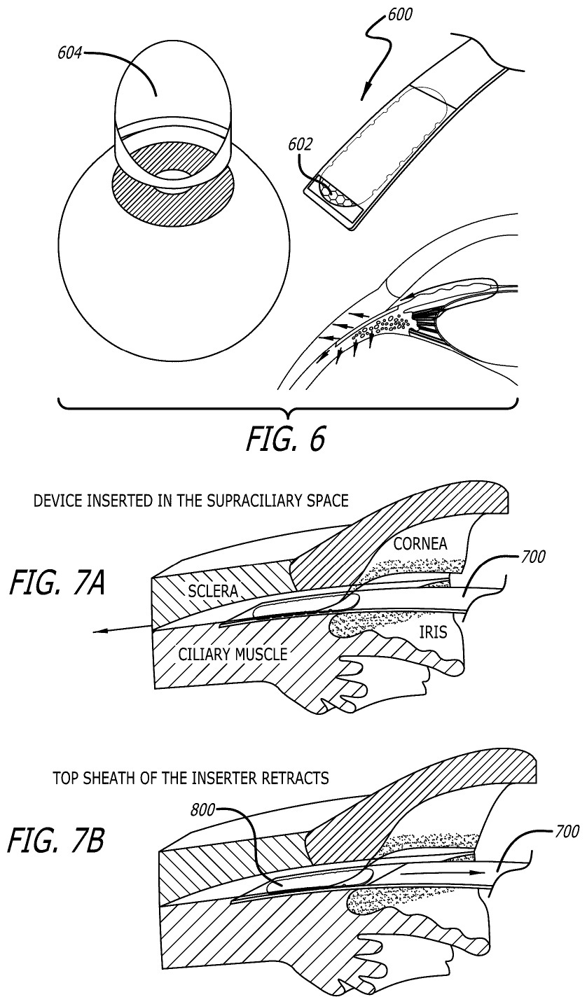

FIG. 6 is a diagram of a treatment device implanted within the supraciliary or suprachoroidal space of the eye, according to an embodiment of the present disclosure. Prior to implanting the treatment device 602 pre-loaded in an insertion device 600 of the present disclosure, a gonioprism 604 may be placed on the patient's eyeball to examine the anterior chamber angle of the eye, which is the area where the iris meets the cornea. This angle is crucial for diagnosing and treating glaucoma and other eye conditions. The gonioprism 604 may be a prism-shaped lens that helps overcome the issue of total internal reflection at the cornea, allowing the ophthalmologist to view this otherwise hidden angle. There are different types of gonioprisms, such as three-mirror and four-mirror lenses, each designed for specific clinical purposes. By placing the gonioprism 604 on the patient's eyeball, as shown in FIG. 6, light from the angle is directed outwards so that it can be visualized through a surgical microscope.

The treatment device 602 is a biocompatible ocular implant that includes a thin, flexible plate to facilitate safe, comfortable, and effective treatment. For example, the treatment device 602 may include a plate structure pre-loaded in an insertion device of the present disclosure, the plate structure having a plurality of channels configured to facilitate the draining of accumulated aqueous in the anterior chamber of the eye to the supraciliary pocket. This enables intraocular pressure from the accumulation of the aqueous in the anterior chamber to be reduced. The removed aqueous in the pocket is gradually reabsorbed by surrounding tissue, which enables further accumulating aqueous to be removed from the anterior chamber. This continuous draining of aqueous (e.g., glaucoma drainage) lowers pressure within the eye and protects the optic nerve. A plurality of redundant channels of the plate structure may prevent single-end clogging by scar tissue. Further, the thin profile of the plate structure hinders tissue erosion and helps ensure patient comfort.

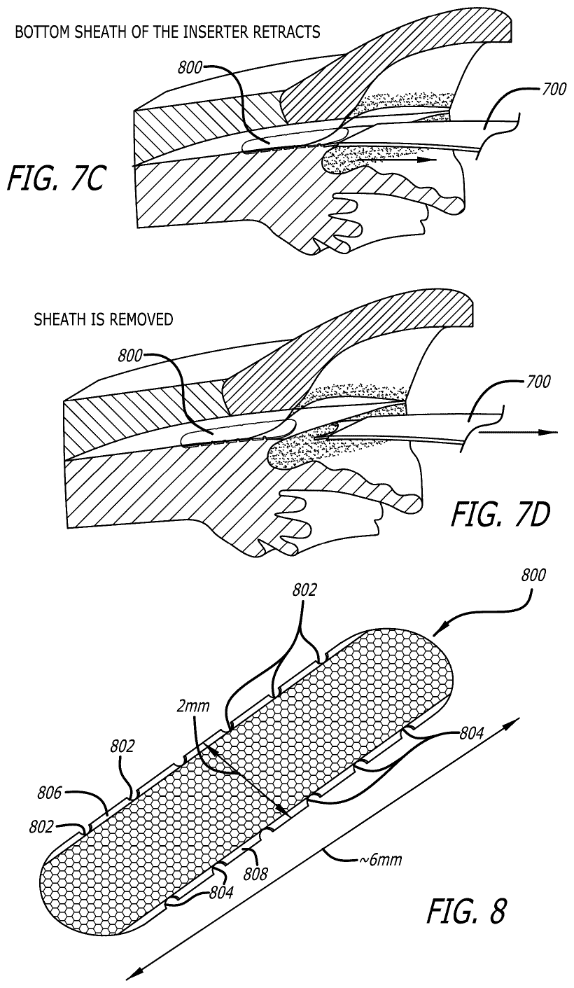

As an example, referring to FIG. 7A, a treatment device loaded in an insertion device 700 of the present disclosure may be inserted in the supraciliary space of an eye. FIG. 7B and FIG. 7C respectively illustrate a top portion and a bottom portion of the treatment device 800 retracting into a user-operated handle of the insertion device 700 sequentially. FIG. 7D shows that the insertion device 700 is removed from a surgical site, leaving the treatment device 800 in place.

### Thin-Film Implant Device Delivery Tool

As will be described fully below, referring to FIGS. 8-12, a device may be configured to achieve an efficient delivery and placement of at least one thin-film implant 800 into supraciliary space to serve as a bleb-less minimally invasive surgery option for glaucoma patients. FIG. 8 illustrates a suture-less plate structure 800, which is similar to the treatment device described above with respect to FIGS. 1-4, 5A, 5B, and 5C, is a biocompatible ocular implant configured to provide to adequate drainage of aqueous humor from an anterior chamber of a patient's eye. According to a preferred embodiment, the plate structure 800 may include a plurality of lateral notches 802, 804 respectively along two perimeters 804, 808 of its thin-film structure to promote fixation via tissue integration. In one example, the plate structure 800 may have a rectangular or oblong shape with a width of 2 mm and a length of 6 mm or so. It should be appreciated that the exact shape and dimension of the plate structure 800 may be determined and implemented based on any desired characteristics.

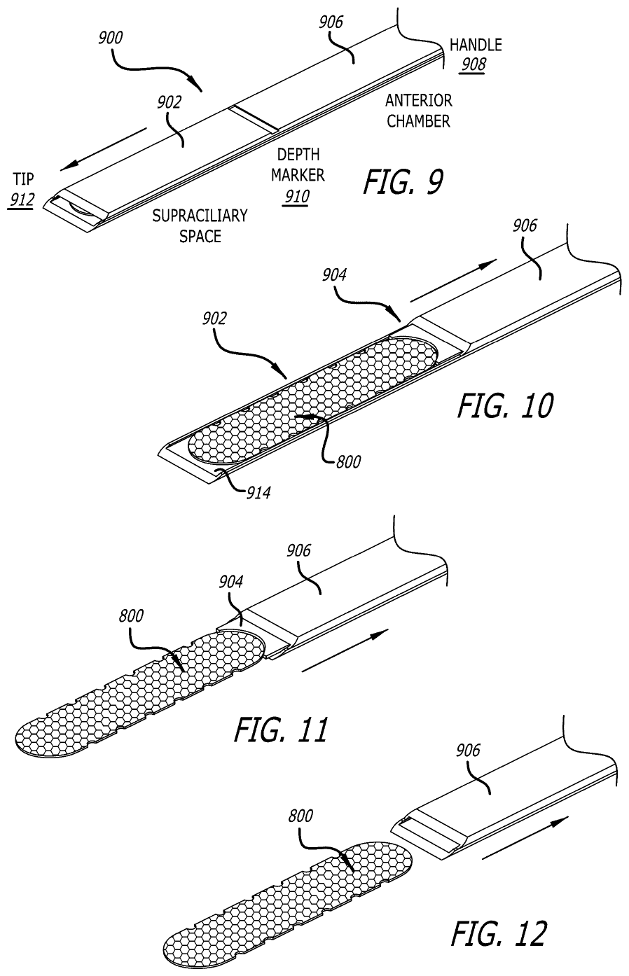

Referring to FIGS. 9-12, an insertion device 900 may include a sheath structure with a plate structure 800 preloaded therein. In one embodiment, the insertion device 900 may have a distal portion 902, a support structure 904, a proximal portion 906, a handle 908 (not shown), and depth marker 910 implemented on the outer surface of the insertion device 900. The distal portion 902 may be sized and dimensioned for housing and protecting the plate structure 800 prior to deployment, as shown in FIG. 8. During use, the insertion device 900 may be positioned ab interno into the supraciliary space of a patient's eye up to the depth marker 910 indicated on the outer surface of the insertion device 900 and a beveled tip 912 of the distal portion 902 may facilitate entry into the space.

A surgeon may manipulate the insertion device 900 under visualization to position the distal portion 902 having the plate structure 800 preloaded therein to a target tissue surface and then retract the distal portion 902 axially with respect to the proximal portion 906 of the insertion device 900. As shown in FIG. 10, as a first step of the deployment process, a top half of the distal portion 902 may be retracted, exposing most or all of the plate structure 800. During this process, the support structure 904 and a bottom half 914 of the distal portion 902 collectively maintain the plate structure 800 in a stabilized, pre-deployment state, and the superior scleral tissue of the patient's eye engages the top surface of the plate structure 800. Thereafter, referring to FIG. 11, the bottom half 914 of the distal portion 902 may be retracted by the surgeon, while the support structure 904 retains the plate structure 800 in place, which is now fully exposed. In one aspect, the support structure 904 may be positioned between the top half and the bottom half 914 of the distal portion 902 and adjacent a proximal end of the plate structure 800 and configured to provide, e.g., a forward pressure axially when the bottom half 914 of the distal portion 902 is retracted in an opposite direction, such that the plate structure 800 remains flat, without wrinkles, creases or folding at the surgical site (e.g., the supraciliary space of the patient's eye), and without significant subsequent manipulation by a variety of other tools. For example, as shown in FIGS. 10 and 11, the support structure 904 may have a side wall and outer surface configured to match surface contours of the proximal end of the plate structure 800 to prevent any axial or lateral movements when the top half or the bottom half 914 of the distal portion 902 slides against the plate structure 800. It should be appreciated that the support structure 904 may use any suitable mechanisms and components to counteract the forces generated by the top half and/or bottom half of the distal portion 902 during retraction while maintaining the flatness of the plate structure 800.

Once the insertion device 900 is determined to be positioned appropriately within the patient's eye, the insertion device 900 is removed from the surgical site, leaving the plate structure 800 at the target tissue surface, as shown in FIG. 12. The plurality of corrugations or lateral notches respectively along two perimeters may keep the plate structure 800 retained in the supraciliary pocket.

As such, the insertion device 900 of the present disclosure may use a two-step deployment process of a preloaded plate structure 800 into a surgical site that is open or minimally invasive (e.g., an ocular surgical site) while protecting the plate structure 800 during insertion and placement and preventing wrinkling due to axial or lateral movements. In one example, the exposing and deploying process of the plate structure 800 may be implemented by installing at least one component (e.g., at least one of a lever, a button, a switch, and a slider) on the handle 908 of the insertion device 900. According to one embodiment, a single lever may be contemplated to retract the top half of the distal portion 902 followed by the retraction of the bottom half 914 of the distal portion 902 in a sequential order. According to another embodiment, two independent levers may be implemented on the handle 908 to respectively control the retractions of the top half and the bottom half 914. In one aspect, a second lever for deploying the plate structure 800 may only be activated after a first lever has retracted the top half of the distal portion 902 of the insertion device 900 at the target surgical site, exposing the plate structure 800. That is, locking and unlocking mechanism (not shown) may be implemented to control the second lever to correspond to a full extended state and a retraction state of the top half of the distal portion 902 of the insertion device 900, respectively.

According to additional embodiments, the channel geometry design of the insertion device 900 may be optimized for the supraciliary space. For example, the bottom outer surface of at least a portion of the insertion device 900 may be configured to have a curvature or shape to match the entry for supraciliary space from the opposite side of cornea. Suitable textures and/or features may be added to the plate structure 800 to depict micro-barbs or similar features that promote retention. In addition to the depth marker 910, the distal portion 902 of the insertion device 900 may be made of transparent materials to allow a direct visualization of the plate structure 800 during insertion and placement. In addition, the insertion device 900 may include insertion check mechanism (e.g., a plurality of notches or marks). For example, one notch may indicate an appropriate insertion depth of the insertion device 900 into the supraciliary space of the patient's eye, whereas a surgeon may consider removing and replacing the insertion device 900 if two or more notches are exposed within the anterior chamber.

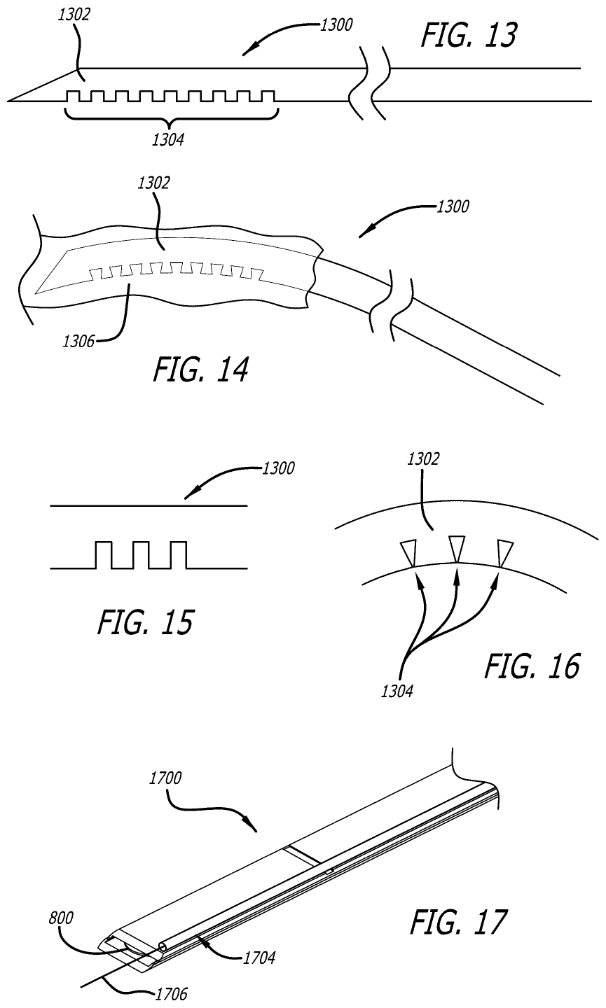

In yet another embodiment, referring to FIGS. 13-16, an insertion device 1300 of the present disclosure may include a series of one or more notches on either or both of the upper and lower portions that render a selected section of the insertion device 1300 flexible and able to automatically conform to the anatomy of the surgical site (e.g., a patient's eye). As shown in FIG. 13, the insertion device 1300 may include a straight shaft and a plurality of notches 1304 implemented on a lower portion of its flexible tip portion 1302. FIG. 14 illustrates that the insertion device 1300 may have a curved shaft and a plurality of notches implemented on a lower portion of a flexible tip portion 1302 of the insertion device 1300 when the insertion device 1300 is inserted into eye tissue 1306. As shown in FIG. 15, a plurality of example notches before flexing may generally be transverse to the longitudinal axis of the shaft of the insertion device 1300 and allow the material of the insertion device 1300 to bend up to a point without buckling or kinking. This may be achieved through the optimal geometry of the individual notch as well as the appropriate placement and spacing of the notches, such that approximately 5-10 mm of the distal portion of the insertion device 1300 is flexible in order to follow the curvature of the human globe, which has a diameter of about 25 mm. In one embodiment, an example notch depth may be about 10-50% of the wall thickness of the shaft of the insertion device 1300. The curvature may be limited upon flexing to the point when the edges of each notch close and thus generate a significant reaction force which resists further bending. FIG. 16 illustrates maximum flexing or closing of multiple notches 1304 to limit the curvature of the tip portion 1302 of the insertion device 1300.

It should be appreciated that any suitable notch designs on the tip portion 1302 of the insertion device 1300 may be implemented depending upon the material used for the tip portion 1302 and the specific application requirements of the insertion device 1300. For example, inverted V-shaped or Chevron notches, semi-circular or U-shaped notches, C-shaped notches, triangular-shaped notches, or zigzag or wavy notches may be implemented in a selected notched region or bending section of the tip portion 1302 to achieve variable flexibility while allowing bending at specific points within the surgical site with minimal impact on the insertion device 1300's structural integrity.

Referring to FIG. 17, in accordance with additional embodiments, an insertion device 1700 of the present disclosure may include at least one lumen 1704 implemented on its outer or inner surface to accommodate a guidewire 1706 to enable the placement of the insertion device 1700 and a treatment device 800 preloaded therein into a surgical site more reliably. The guidewire 1706 may be a thin and flexible wire made of e.g., stainless steel or nitinol, and used to guide the insertion device 1700 through the surgical site. As such, the guidewire 1706 acts as a pathfinder to enable navigation of complex and tortuous anatomical structures (e.g., a patient's eye) while minimizing trauma to the surrounding tissue. The guidewire 1706 may vary in stiffness, flexibility, coating (hydrophilic for better glide), tip shape (e.g., floppy or stiff), and size, depending on the specific surgery conditions. The guidewire 1706 may be deployed through an access point, such as an incision, in advance of the insertion of the insertion device 1700 and may be optimally positioned by trial-and-error with minimal trauma. In some embodiments, the guidewire 1706 may be steered using imaging techniques like gonioscopy, ultrasound biomicroscopy, or anterior segment optical coherence tomography, allowing a surgeon to safely navigate around bends or obstructions without causing damage to ocular tissues. Moreover, different tips on the guidewire 1706 (e.g., soft, floppy, or more rigid) may allow the surgeon to negotiate curves or challenging sections of anatomy. Once properly positioned at the appropriate surgical site in the eye, the lumen 1704 may be loaded onto the proximal end of the guidewire 1706 and positioned at the target location by following the guidewire 1706 into place. With the guidewire 1706 in place, the insertion device 1700 and a treatment device 800 preloaded therein may be delivered via the lumen 1704 to the target area. According to certain embodiments, the lumen 1704 may also allow for injection of contrast dye or saline to aid visualization, or the delivery of therapeutic agents such as medications. The lumen 1704 may be appropriately sized to allow smooth passage of the guidewire 1706 and other instruments without excessive friction. The lumen 1704 may be flexible and durable to minimize resistance and ensure smooth advancement. As such, the guidewire 1706 provides a precise and minimally traumatic pathway for advancing the insertion device 1700, while the lumen 1704 implemented on the insertion device 1700 ensures smooth passage of the guidewire 1706, facilitating accurate navigation and targeted treatment delivery.

Although the present technology has been described in detail for the purpose of illustration based on what is currently considered to be the most practical and preferred implementations, it is to be understood that such detail is solely for that purpose and that the technology is not limited to the disclosed implementations, but, on the contrary, is intended to cover modifications and equivalent arrangements that are within the spirit and scope of the appended claims. For example, it is to be understood that the present technology contemplates that, to the extent possible, one or more features of any implementation can be combined with one or more features of any other implementation.

Unless otherwise indicated, all numbers expressing quantities, properties, and so forth used in the specification and claims are to be understood as being modified in all instances by the term “about.” Accordingly, unless indicated to the contrary, the numerical parameters set forth in the specification and attached claims are approximations that may vary depending upon the desired properties sought to be obtained by the present disclosure. At the very least, and not as an attempt to limit the application of the doctrine of equivalents to the scope of the claims, each numerical parameter should at least be construed in light of the number of reported significant digits and by applying ordinary rounding techniques. Notwithstanding that the numerical ranges and parameters setting forth the broad scope of the disclosure are approximations, the numerical values set forth in the specific examples are reported as precisely as possible. Any numerical value, however, inherently contains certain errors necessarily resulting from the standard deviation found in their respective testing measurements.

The terms “a,” “an,” “the” and similar referents used in the context of describing the disclosure (especially in the context of the following claims) are to be construed to cover both the singular and the plural, unless otherwise indicated herein or clearly contradicted by context. Recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein is intended merely to better illuminate the disclosure and does not pose a limitation on the scope of the disclosure otherwise claimed. No language in the specification should be construed as indicating any non-claimed element essential to the practice of the disclosure.

Groupings of alternative elements or embodiments of the disclosure disclosed herein are not to be construed as limitations. Each group member may be referred to and claimed individually or in any combination with other members of the group or other elements found herein. It is anticipated that one or more members of a group may be included in, or deleted from, a group for reasons of convenience and/or patentability. When any such inclusion or deletion occurs, the specification is deemed to contain the group as modified thus fulfilling the written description of all Markush groups used in the appended claims.

Certain embodiments of this disclosure are described herein, including the best mode known to the inventors for carrying out the disclosure. Of course, variations on these described embodiments will become apparent to those of ordinary skill in the art upon reading the foregoing description. The inventor expects skilled artisans to employ such variations as appropriate, and the inventors intend for the disclosure to be practiced otherwise than specifically described herein. Accordingly, this disclosure includes all modifications and equivalents of the subject matter recited in the claims appended hereto as permitted by applicable law. Moreover, any combination of the above-described elements in all possible variations thereof is encompassed by the disclosure unless otherwise indicated herein or otherwise clearly contradicted by context.

Specific example embodiments disclosed herein may be further limited in the claims using consisting of or and consisting essentially of language. When used in the claims, whether as filed or added per amendment, the transition term “consisting of” excludes any element, step, or ingredient not specified in the claims. The transition term “consisting essentially of” limits the scope of a claim to the specified materials or steps and those that do not materially affect the basic and novel characteristic(s). Example embodiments of the disclosure so claimed are inherently or expressly described and enabled herein.

In closing, it is to be understood that the embodiments of the disclosure disclosed herein are illustrative of the principles of the present disclosure. Other modifications that may be employed are within the scope of the disclosure. Thus, by way of example, but not of limitation, alternative configurations of the present disclosure may be utilized in accordance with the teachings herein. Accordingly, the present disclosure is not limited to that precisely as shown and described.

1. An insertion device, comprising:
a distal portion configured to enclose and protect a thin-film implant device during delivery and placement into a surgical site, wherein the distal portion includes a top half and a bottom half, and the thin-film implant device is placed between the top half and the bottom half; and
a support structure positioned between the top half and the bottom half and adjacent a proximal end of the thin-film implant device,
wherein the top and bottom halves are retractable independently of one another.

2. The insertion device of claim 1, further comprising a handle portion.

3. The insertion device of claim 1, further comprising a marker configured to indicate an insertion depth of the insertion device into the surgical site.

4. The insertion device of claim 2, wherein the handle portion is configured to include a component to retract the top half of the distal portion followed by a retraction of the bottom half in a sequential order, wherein the component includes at least one of a lever, a button, a switch, and a slider.

5. The insertion device of claim 2, wherein the handle portion is configured to include two levers to control retractions of the top half and the bottom half of the distal portion independently.

6. The insertion device of claim 1, wherein the support structure is configured to prevent axial and lateral movements of the thin-film implant device after implantation.

7. The insertion device of claim 1, wherein the thin-film implant device includes a plurality of lateral notches implemented along at least a portion of perimeters of the thin-film implant device to promote fixation via tissue integration.

8. The insertion device of claim 1, wherein a bottom outer surface of the insertion device is configured to have a curvature or shape to match a curvature of the surgical site.

9. The insertion device of claim 1, wherein the thin-film implant device includes micro-barbs or features configured to promote retention.

10. The insertion device of claim 1, wherein the distal portion of the insertion device is made of transparent materials to allow a direct visualization of the thin-film implant device.

11. The insertion device of claim 1, wherein the thin-film implant device has a visual marker at one end to indicate a proper depth of insertion within the surgical site.

12. The insertion device of claim 1, wherein a tip of the distal portion of the insertion device is beveled to facilitate penetration of tissues into the surgical site.

13. The insertion device of claim 1, wherein a selected section of the distal portion comprises a plurality of notches implemented on an outer surface of at least one of the top half and the bottom half.

14. The insertion device of claim 13, wherein the plurality of notches are configured to flex to allow the distal portion to bend up to conform to an anatomy of the surgical site during the delivery and placement of the thin-film implant device into the surgical site.

15. The insertion device of claim 14, wherein each of the plurality of notches is configured to flex within a range to control a curvature of the selected section.

16. The insertion device of claim 13, wherein a longitudinal length of the selected section is between 5-10 mm.

17. The insertion device of claim 1, further comprising a lumen implemented on an outer or inner surface of the insertion device to accommodate a guidewire positioned at a selected location within the surgical site to facilitate placement of the insertion device.
